(19) United States
(12) Patent Application Publication
(10) Pub. No.: US 2006/0247526 A1
(43) Pub. Date: Nov. 2, 2006

(54) Distance measurement method and device using ultrasonic waves

(76) Inventors: Dong Hwal Lee, Busan (KR); Heui Tay An, Busan (KR); Young Shin Kang, Busan (KR)

Correspondence Address: IPLA P.A., 3580 WILSHIRE BLVD., 17TH FLOOR, LOS ANGELES, CA, 90010, US

(21) Appl. No.: 10/566,286

(22) PCT Filed: Jul. 29, 2004
(86) PCT No.: PCT/KR04/01917

§ 371 Date: Jan. 27, 2006

(30) Foreign Application Priority Data
Jul. 29, 2003 (KR) 10-2003-0052239

(51) Int. Cl. A61B 8/00

(52) U.S. Cl. 600/438

(57) ABSTRACT

The present invention provides a distance measurement method and device using ultrasonic sufficiently amplifying a received ultrasonic wave signal and separating a specific frequency from an ultrasonic wave signal mixed with an unnecessary signal to extract an arrival signal of a first pulse. It is thus possible to calculate a distance of an object safely.

### TECHNICAL FIELD

The present invention relates to a distance measurement method and device using ultrasonic. More particularly, the present invention relates to a distance measurement method and device using ultrasonic wherein a distance is measured by transmitting and detecting an ultrasonic signal so that it is applied to various distance measurement systems, localization system, factory automation (FA), a mobile robot, pseudolite, etc.

### BACKGROUND ART

A distance measurement method may be classified depending on a contact or non-contact type, the distance range, its use and the like. The distance measurement method may be classified into a long or short distance measurement method and a precise distance measurement method depending on the range of a distance. The long-distance measurement method may include a laser method, a RF method, an IR method, an ultrasonic method, a CCD method, a scale method and so on. The short distance measurement method may include an induced current method, a photo sensor and a laser sensor. The precise distance measurement method may include an eddy current sensor, a magnetic sensor, a LVDT sensor and a linear scale and the like.

Meanwhile, distance measurement in the field where a high performance localization must be performed in a wide area such as a multi-body mobile robot must be implemented in a non-contact method A multi-body must be recognized respectively at the same time and a distance detection of at least several tens of meters range must be enabled Furthermore, it is required that the distance measurement be implemented inexpensively and meets requirements such as good detection, stability, real-time and indoor measurement.

The distance measurement method that have been proposed so far in localization area do not meet above requirements. That is, in order to meet above requirements, the distance measurement method in localization area must be able to measure farther distances. In viewpoint of the performance, a laser is the most preferred one but has problems in viewpoint of cost and safety. The pseudolite using RF has problems in that it requires high technology and high cost. In viewpoint of cost, the ultrasonic or IR method is preferred but still has a problem in that its performance is lower than the laser and the RF method

Meanwhile, an air-borne ultrasonic sensor has been widely applied to many research and many industrial applications in spite of a limitation to its performance. This air-borne ultrasonic method has been widely applied to a sensor for collision avoidance or object detection in a mobile robot, a detector for detecting an object in the rear of a car, a traffic detector, a speedometer, a telemeter for construction, a security sensor for detecting invasion of animals or mankind and the like.

The distance measurement method using ultrasonic measures time between an arrival time (T1) and a starting time (T0) of the ultrasonic signal when an ultrasonic is transmitted toward a target. In the above, the arrival time is a time taken by the ultrasonic signal that generates from transmitter and arrives to receiver. If an travel time T1 to T0 is calculated, and then multiplied by the speed of sound, it becomes a distance to the target.

### DISCLOSURE OF INVENTION

### Technical Problem

Currently, the most typical method in distance measurement using ultrasonic is a threshold method, as shown in FIG. 1.

FIG. 1 is a graph illustrating a state where a first zero crossing is detected in a received signal. This method is to measure a distance by detecting an ultrasonic signal of over a threshold level. This typical distance measurement method using such ultrasonic, however, has a difficulty due to several uncertainty.

In FIG. 1, there are shown two error causes in finding a time T1 when an ultrasonic is reflected from a target and then returns to an original point where the signal is transmitted in a threshold method (i.e., T2 includes two error sources). In the above, a received waveform 10 is detected from the background of ambient noise having a noise (RMS) amplitude level 12. In this threshold method, a received signal is rejected until the amplitude of the received signal exceeds a threshold value 14 that is significantly higher than ambient noise in order to guarantee whether ultrasonic that are actually received are echo pulses not noise. Therefore, the conventional method using the threshold value employs a time T2 of a zero crossing 16 that exceeds the noise amplitude level 12 and follows the first detection amplitude as a measurement value of T1. The time T2 is the starting point that is to be found by the time T1 among the received signal. In such a threshold method, T2 is acquired since only a signal of over a threshold value is recognized Accordingly, there occur errors in distance measurement as much as T2−T1. These errors are further significant when noise is mixed with the received signal.

Another error source is the fact that the speed of sound through a propagation medium can vary unpredictably. For example, in the case where a propagation medium is air, the speed of sound depends on an atmospheric pressure, a temperature and humidity in a propagation path. If the atmosphere is uniform in the propagation path of the sound, a measurement device can compensate for a variation in the pressure, temperature and humidity using pressure, temperature and humidity sensors. It is, however, difficult to guarantee an uniform distribution of the propagation medium Therefore, in this threshold method, the transmitted signal strength in air is weakened severely according to ultrasonic characteristics. Thus, the attenuation becomes higher as the distance becomes farther, and the amount of the signal becomes smaller depending on the distance. It is thus impossible to detect the signal itself. As a result, there are lots of limitations to the distance of detection.

Meanwhile, FIG. 2 shows technology proposed in U.S. Pat. No. 5,793,704 entitled ‘Method and Device for Ultrasonic Ranging’ by David Freger, Ashkelon Islael, which discloses an envelope detecting method for measuring a distance by extracting a maximum amplitude ultrasonic signal in order to detect the aforementioned threshold value and improve the problems. More particularly, FIG. 2 shows three received waveforms that are propagated through the medium (the atmosphere) of different propagation velocity for the same target and are then received by a receive circuit. This shows the basic principle of conventional technology.

Referring to FIG. 2, the received ultrasonic signal has a constant envelope regardless of the strength of a signal. Such a conventional envelope detecting method acquires the characteristic of the envelope. In this method, as the received signal is not saturated although the distance is near, the envelope is always acquired. The start point of the received waveform is detected using a maximum amplitude point of the envelope obtained thus through back-tracking. This method enables a more accurate start point T2 to be found compared to the threshold value mode.

If the signal is saturated, however, this method has a problem that the envelope is not obtained. It is thus required that means for controlling the gains of transmitting and receiving amplifiers as a variable structure be added.

Referring back to FIG. 2, an upper received waveform 20 reflects back through ‘fast’ air, an intermediate received waveform 30 reflects back through ‘average’ air, and a lower received waveform 40 reflects back through ‘slow’ air.

Generally, an ultrasonic wave sensor responds to the onset of ultrasonic wave energies non-linearly in order to detect an ultrasonic signal reflecting back regardless of a piezoelectric type or a magnetic type. This sensor is characterized in that its response time is shorter in a high energy signal than in a low energy signal. Moreover, the energy level of the received waveform propagated through air tends to vary contrary to the propagation velocity. That is, with respect to a predetermined propagation energy level, the received waveform that reflects back through ‘slow’ air tends to have higher energy than the received waveform that reflects back through ‘fast’ air.

Therefore, first arrival times of respective waveforms (pulses; 20, 30 and 40) are quite different as shown in FIG. 2. In this case, the lower received waveform 40 has higher energy than the intermediate received waveform 30 and the intermediate received waveform 30 has higher energy than the upper received waveform 20. Accordingly, a receiver sensor responds to the lower received waveform 40 more rapidly than the intermediate received waveform 30 and responds to the intermediate received waveform 30 more rapidly than the upper received waveform 20. At this time, an envelope 22 of the amplitude envelope (the upper received waveform 20), an envelope 32 of the intermediate received waveform 30 and an envelope 42 of the lower received waveform 40 of respective received waveforms have maximum values, respectively, approximately at the same time. Thus, distance measurement based on picking of a maximum value of the amplitude envelope of the received waveform is relatively less influenced by variation in the speed of sound. According to this conventional method, since the maximum amplitude far exceeds a standby noise level, it is possible to avoid error related to picking of the first zero crossing of the received waveform

In the method and device for measuring a distance by extracting the maximum amplitude signal of the ultrasonic waves, if the received signal of the sensor is amplified, noise is mixed with the signal. Thus, there is a problem in that distance measurement is impossible since a detection signal becomes weak in case of the range of 3˜10 m. In other words, in this conventional method, response of a sensor varies depending on the type of the sensor. Thus, it is necessary to modify this method by propagating an ultrasonic wave pulse toward a correction target of a known distance and measuring a time of the maximum amplitude of the received waveform. This correction includes measurement of an amplitude level at a receiver circuit that does not reach a saturation level a little in each correction distance. A table of an optimum amplitude level for this distance is thus provided. If a received waveform signal received alternately is saturated, means for measuring and feedbacking the amplitude is provided in order to reduce the degree of amplification of the receiver. Furthermore, in an actual use, the distance measurement device measures a distance for a target using two or more propagation pulses. In this case, the first pulse is used to obtain rough estimation of the distance for the target and the second pulse is transmitted in order to measure an actual distance.

Therefore, such a conventional envelope method has problems in that it has a complication device construction and lots of limitations in the use. This mode also has a difficulty in application to accurate positional recognition in a wide region since a distance to be measured is short and accuracy is low.

### Technical Solutions

Accordingly, the present invention has been made in view of the above problems, and it is an object of the present invention to provide a distance measurement method and device using ultrasonic waves wherein a distance can be effectively measured without being affected by a limit condition depending on surrounding environment such as the atmosphere and noise in measuring the distance using an ultrasonic wave signal.

### Advantageous Effects

According to the present invention described above, the fact that the frequency properties of noise and an ultrasonic are different is employed. Thus, since it is not affected by the amount of noise, there is no limitation in amplification of a signal. It is thus possible to amplify the signal containing noise sufficiently.

Furthermore, noise and a weak ultrasonic signal are amplified together and a specific frequency of the ultrasonic signal is separated from a sufficiently strong signal and a first signal among the specific frequency is restored. Therefore, even in a weak signal depending on long-distance measurement, it is possible to measure an ultrasonic signal regardless of noise.

Moreover, it is possible to maintain a maximum differential gain regardless of the amount of a received signal. It makes the device simple. Therefore, the present invention has advantages in that long-distance measurement is possible since a weak signal can be amplified sufficiently and the accuracy can be increased since an initial signal lost can be restored.

### DESCRIPTION OF DRAWINGS

Further objects and advantages of the invention can be more fully understood from the following detailed description taken in conjunction with the accompanying drawings in which:

FIG. 1 is a graph for explaining a conventional distance measurement method using ultrasonic waves wherein signals of over a predetermined size are extracted;

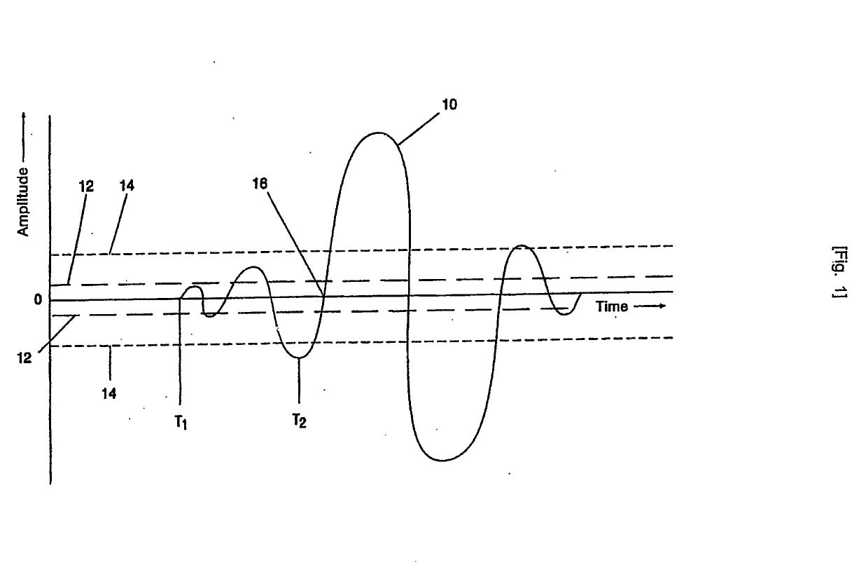

FIG. 2 is a graph for explaining a conventional distance measurement method using ultrasonic waves wherein signals of a maximum amplitude are extracted;

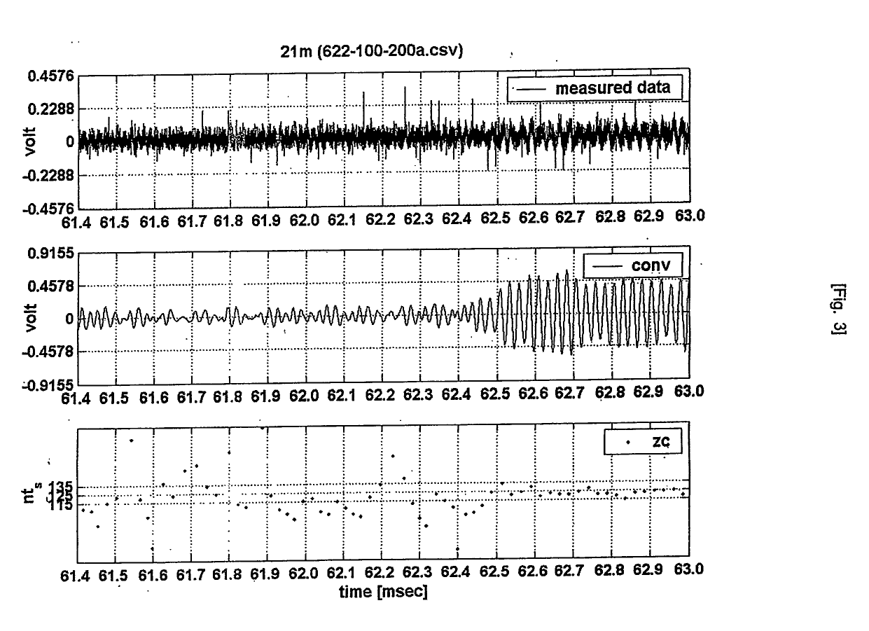

FIG. 3 sequentially shows ultrasonic wave signals that are processed step by step by means of a distance measurement method using ultrasonic waves according to the present invention;

FIG. 4 and FIG. 5 are graphs for explaining a distance measurement method using ultrasonic waves according to the present invention;

FIG. 6 is a block diagram illustrating the construction of a distance measurement device using ultrasonic waves according to a preferred embodiment of the present invention;

FIG. 7 shows received ultrasonic waveforms before they are experienced by a digital signal processing at different distances by means of a distance measurement method and device using ultrasonic waves according to a preferred embodiment of the present invention; and

FIG. 8 shows results that signals are repeatedly measured and processed at the same point of 20 m or more by means of a distance measurement method and device using ultrasonic waves according to a preferred embodiment of the present invention .

### BEST MODE

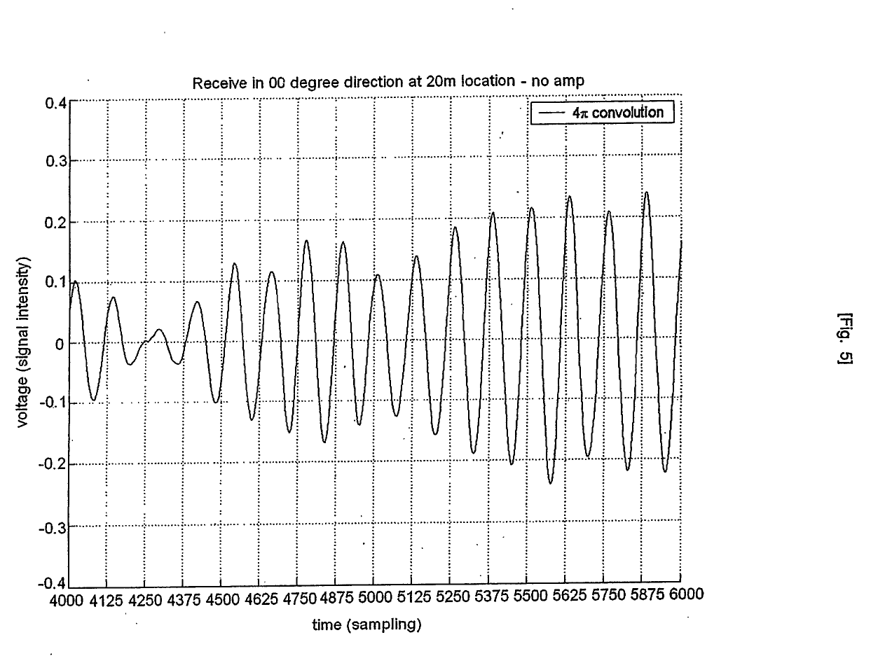

Hereinafter, a preferred embodiment of the present invention will be described in detail with reference to FIGS. 4 to 8. Meanwhile, in the description according to a preferred embodiment of the present invention, description on the basic principle of distance measurement using ultrasonic waves and the construction and acting effects of it that is known to those skilled in the art will be omitted.

FIG. 4 and FIG. 5 are graphs for explaining a distance measurement method using ultrasonic waves according to the present invention. In the above, FIG. 4 is a graph showing a received waveform (v(k)) received at 21.6 m and FIG. 5 is a graph showing waveforms (w(k)) being a result of convolution-processing the received waveform in FIG. 4.

Referring to FIG. 4 and FIG. 5, a waveform shown seen in FIG. 4 is a received signal containing distance information to be measured before or after a specific time. The amount of this received signal is a weak signal similar to nose that cannot be identified by a conventional method such as maximum amplitude detection. Therefore, though it is possible to presume sensually that there is a signal within noise, what point will be defined is full of ambiguities. As a result, the received waveform cannot be processed by an existing method since its signal level becomes smaller than the amplitude of noise. This signal is received when a distance between a transmitter and a receiver becomes far.

If this signal is processed by the present invention, it becomes possible to identify the period of the frequency, as shown in FIG. 5. A waveform shown in FIG. 5 is a result that the received waveform shown in FIG. 4a is digitalized. The waveform includes two kinds of waveforms whose periods are different around a sampling sequence central point 5000th. That is, after the central point, a waveform having a certain period continues, whereas before the central point, the waveform varies irregularly. In the present invention, a first signal is estimated from ultrasonic signals extracted by this method, a time delayed from a time of a signal that is transmitted for the first time is measured and is then converted into a distance.

In this embodiment, the ultrasonic employ direct waves with separated transmission and reception. Generally, the ultrasonic waves employ a reflection wave mode from the object that uses a transducer of a type in which a transmitter and a received are integrated. This integration type has advantages that it has a simple construction and can be easily manipulated. In this integration type, it is difficult to point out an accurate subject point since the beam width becomes wide due to a problem in orientation. It is also difficult to avoid error in measurement from a reflection wave due to interference of an object. Moreover, the reflection type is useless in independently recognizing a location such as a multi-body mobile robot. Therefore, in this embodiment, a transmitter and a receiver are separated each. This separate type may solve several problems mentioned above. In this embodiment, however, as a sensor can be separated, synchronization is made through wires. This may be easily replaced to IR and RF modes.

In this embodiment, in transmitting the ultrasonic, a signal of a specific frequency is amplified so that it is compatible with a design characteristic of a transmitter. In this embodiment, a pulse of 8 periods is amplified and is then input to the transmitter. Of course, an outgoing wave may not be really the 8 periods. If the wave is shorter than the 8 periods, it is disadvantage in separation from noise. There will be a problem that if the wave is longer than the 8 periods, a distance measurement response time becomes long that much.

In this embodiment, a method used to find specific frequency components to be detected includes detecting a sine wave reference waveform of a period to be found and a received waveform through the following convolution operation.
u(k)=sin (x),
where 0<x<4π.
v(k)=rx(k),
where rx is a received signal.

$$w ( k ) = {\sum }_{j} u ( j ) v ( k + 1 - j )$$

In the present invention, it is possible to separate a waveform from noise of other causes including noise from switching power supply that is introduced from a circuit through such convolution operation.

The distance measurement method using the ultrasonic according to the present invention includes digital-signal processing a received signal and then analyzing a trend that frequency components are consistently maintained. By doing so, although the same frequency is detected, response to noise of a similar signal is not made based on determination about whether the frequency is consistent. That is, it can be said that the ultrasonic signal keeps constant but noise is irregular in variation in its frequency and rarely keep consistent in its frequency. Therefore, although the received signal is sufficiently amplified and noise is also amplified, a desired signal of a specific period that does not respond to noise can be extracted.

After a waveform having a specific period of the ultrasonic signal transmitted thus is extracted, each zero crossing is found and is then converted to one period value. Then, a signal that continues over 8 periods with the a signal level to be detected is searched in real time from the periodic value. If the search is completed, the start point of the 8 periods is determined as T2 and a duration, time of flight, TOF=T2−T1 is found If the time is converted to a distance through temperature compensation, a final measurement value is obtained. The temperature compensation can be made through the following operation.
v_{sound}(Temp)=331.5+0.60714×Temp
TOF=T2−T1
d=v_{sound}(Temp)×TOF

Meanwhile, in the present invention, as a signal responds to a weak signal, the direct wave is a signal that will be measured by a first arrival signal. In the event a reflection wave is employed, signals from objects scattered around an object to be measured can be sensed. Therefore, it is required that a signal within an identifiable range be disregarded. To this end, a distance range that will be excluded from the measurement according to the aforementioned embodiment is specified and a distance that exceeds the specified range is then measured.

Furthermore, according to the present invention, since a signal is extracted using the frequency, a changed frequency is received if a moving object is to be measured. It is thus required to adopt this change. Accordingly, the present invention can apply the frequency to be separated variably considering that the frequency of an ultrasonic wave pulse received is changed if a moving object is the object of measurement.

In addition, a distance to be measured usually is dependent on the speed that ultrasonic propagates within the medium. At this time, the medium may be changed depending on various factors such as temperature. Therefore, in the present invention, in order to measure the velocity of sound of the medium that the ultrasonic propagates, an ultrasonic receiver is installed at a known position to measure the arrival time of a received signal for an ultrasonic that is sent at the same time. The ultrasonic is then received at a location where a measurement value needs to be found, thus measuring a distance. Thereby, a more stable result can be obtained. Generally, a sound wave is affected by the medium. If distance measurement using ultrasonic is applied to a simple application, this fact is not considered as the object of consideration itself. As in the present invention, if the degree and the arrival distance are improved, the sound wave is affected in proportion.

Therefore, in the present invention, a path along which a similar medium passes is measured for an object to be measured and its result is reflected. For this, if a receiver is disposed at a known distance and an arrival time is measured, the velocity of sound can be known in advance depending on atmospheric environment between objects to be measured. That is, the velocity of sound refers to an arrival distance that is divided by time. Thus, if the arrival distance and time are known, the state of the medium on a path to be measured can be known.

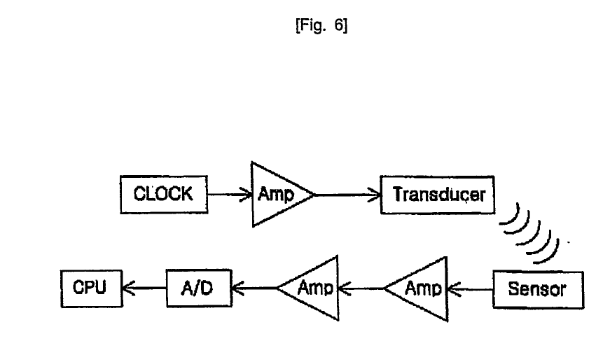

FIG. 6 is a block diagram illustrating the construction of a distance measurement device using ultrasonic according to a preferred embodiment of the present invention.

Referring to FIG. 6, the distance measurement device using ultrasonic according to a preferred embodiment of the present invention includes a 40 KHz ultrasonic sensor. A received waveform is sampled into 5 MHz and is then displayed. In FIG. 6, the concept of the distance measurement device using ultrasonic in short is shown schematically. In the concrete, the device according to this embodiment includes an ultrasonic transmitter, a sensor, an amplifier, an analog filter, a secondary amplifier, an A/D converter, a memory, a digital signal processor, a display unit, a numerical input unit and a communication unit.

In this device, transmission of the ultrasonic includes transmission of 8 pulses of a specific period and the received ultrasonic signal is amplified twice by the amplifier. The differential gain is limited since it is accompanied by oscillation. Therefore, the amplified signal passes through a filter that attenuates other frequency in order to protect a specific frequency of the signal at its maximum and is then sufficiently amplified again. The signal amplified again thus is converted into a digital signal through the A/D converter so that it can be processed in a digital computer with the memory, the digital signal processor, the display unit, the numerical input unit and the communication unit. Furthermore, the digital signal processor that extracted the specific frequency from the ultrasonic wave signal may be used in moving averages, convolution and numerical analysis such as FFT.

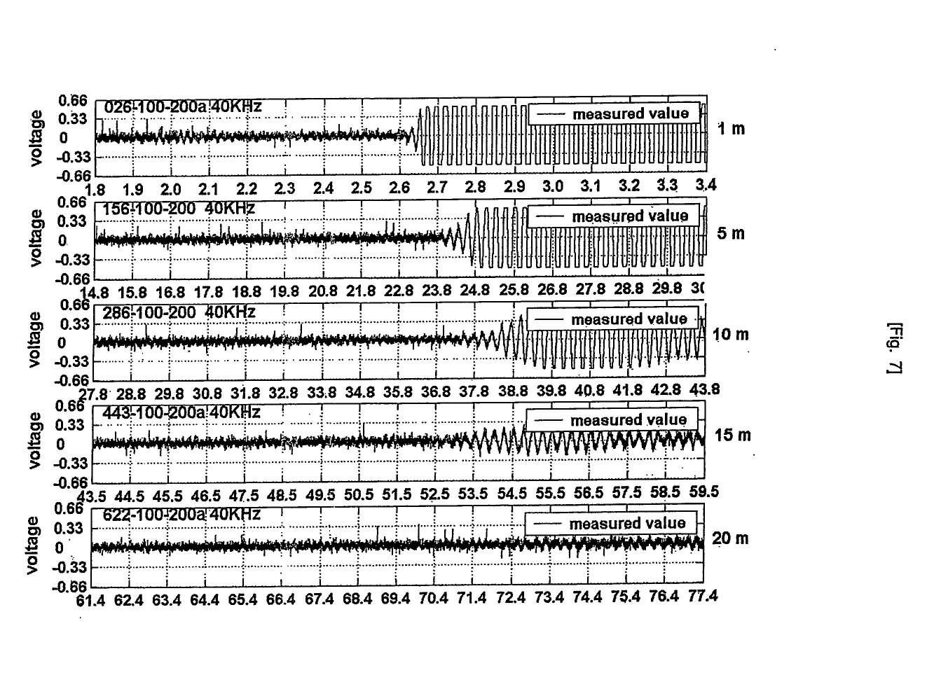

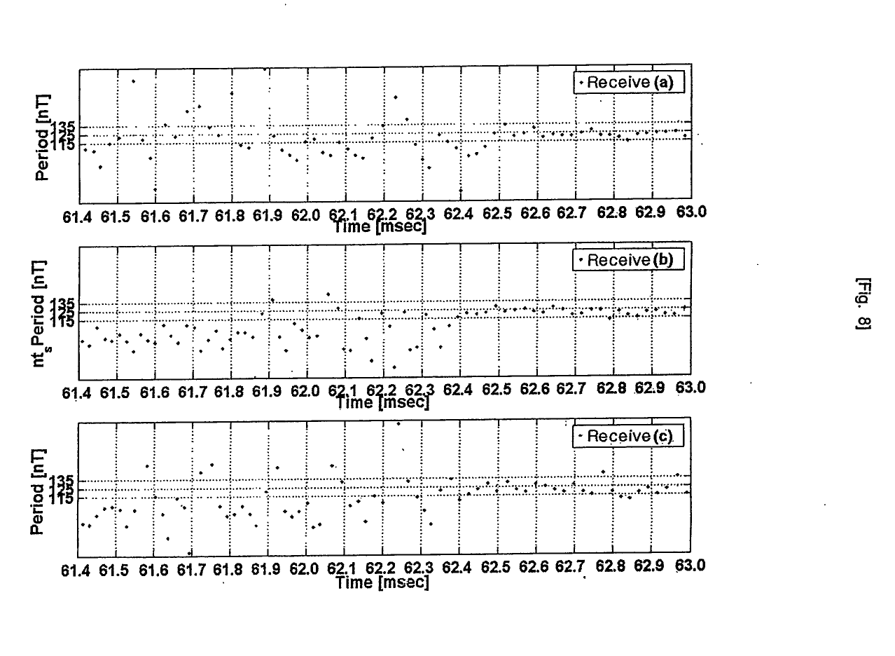

FIG. 7 is graphs showing received ultrasonic waveforms before they are experienced by a digital signal processing at different distances by means of a distance measurement method and device using ultrasonic waves according to a preferred embodiment of the present invention. FIG. 8 is graphs showing results that signals are repeatedly measured and processed at the same point of 20 m or more by means of a distance measurement method and device using ultrasonic waves according to a preferred embodiment of the present invention.

A performance experiment on the method and device according to a preferred embodiment of the present invention was made with sensors for transmission and reception separately disposed and was made in indoor corridor environment. Further, the experiment has been made under a condition with no car driving or special noise to be considered that may affect external sound wave interference.

Such an experiment was made by a method wherein ultrasonic are transmitted, a receiver receives and samples its signal digitally, the sine wave of a specific period, convolution and a zero crossing point are calculated, a crossing point time is continually stored as a periodic value, whether a specific period continues for 8 periods is determined, if it is determined that the specific period continues for the 8 periods, the search is finished, TOF is calculated, temperature is compensated for and is then converted to a distance.

FIG. 7 shows results of the received ultrasonic waveform before it is experienced by a digital signal processing for distances 1 m, 5 m, 10 m, 15 m and 20 m. As shown in FIG. 7, in case of a near one, the gain of the amplifier is almost the same as a level in which the received signal is saturated At this time, the method according to the present invention is not a method using a level. Thus, there is an advantage in that normal measurement is possible even in case of saturation. In other words, through the method for comparing the levels, it is possible to identify the level that is not operated by a noise signal up to 10 m in one scale (within the range of 1/2 in FIG. 7). It is, however, impossible to identify the level in 15 m and 20 m. Meanwhile, if the amount of a level that becomes a reference is set low, the level responds to a noise signal. Thus, the amount of the level to concede is decided for a stabilized operation.

FIG. 8 shows a result that the signal is repeatedly measured three times at the same point processed according to the present invention at a distance of over 20 m.

As shown in FIG. 8, the vertical axis in the graph indicates a time dimension of a unit where a sampling period is 1. This indicates the period of each waveform Therefore, the height of 125 scales indicates a 40 KHz period Therefore, according to the present invention, it is shown that a signal to be detected is generally strong among noise. In view of the frequency, the same frequency components are shown here and there. A signal component that keeps the frequency is represented in a level that is clearly discriminated compared to the noise. In addition, the start point of the signal represents a tendency. The start point of the signal corresponds to the accuracy. in case of a and b in the graph, the arrival of the signal can be recognized at a point of inflection. At the same position, in case of c, it is possible to recognize a point where there is a symptom on which a boundary point by the signal and noise may be estimated. In addition, measurement values repeated three times are 21.634 m, 21.633 m and 21.632 m, respectively, and the repeatability is ±1 mm. This shows that in the distance measurement method and device using ultrasonic according to the present invention, the repeatability at a distance of 20 m or more is 2mm/20 m, i.e., 1/10,000 resolution.

In order to confirm the present invention, an experiment is performed in a laboratory of a range of about 20 m. In selecting a sensor, if the frequency is changed from 40 KHz to 20 KHz and the signal output level is increased, distance measurement of a range close to 100 m is possible.

As such, according to the distance measurement method and device using ultrasonic of the present invention, it is possible to measure stably a distance region that cannot be measured by conventional method and device and to expand its distance range.

### MODE FOR INVENTION

To achieve the above object, according to the present invention, there is provided a distance measurement method using ultrasonic waves, including the steps of: transmitting an ultrasonic wave pulse having a specific frequency to an object; receiving the ultrasonic wave pulse that is reflected from the object or directly transmitted; and extracting a specific frequency of the received signal to find an arrival time of a first pulse and converting the time into a distance.

In the distance measurement method using the ultrasonic waves, the step of finding the arrival time and converting the time into the distance further includes the step of separating a specific frequency of the ultrasonic wave pulse and converting an arrival time of an ultrasonic wave pulse that is received for the first time among the separated ultrasonic wave pulses into the distance, in a state where the waveform is mixed with noise having different frequency properties from the specific frequency of the transmitted ultrasonic waves.

In the distance measurement method using the ultrasonic waves, in the step of converting the time into the distance, the extraction of the specific frequency from the received ultrasonic wave pulse further includes the steps of: amplifying the received ultrasonic wave pulse to generate an amplified signal; weakening a signal of an unnecessary frequency among the amplified signal through an analog filter circuit to generate a filtered signal; amplifying the filtered signal again to generate a re-amplified signal; converting the re-amplified signal into a digital signal; and extracting a specific frequency from the converted digital signal through a digital signal processing.

The distance measurement method using the ultrasonic waves further includes the step of specifying a distance range to be excluded when measuring a distance of the object, so that a distance exceeding the specified distance range is measured

In the distance measurement method using the ultrasonic waves, the step of receiving the ultrasonic wave pulse reflected from the object while the object is moving comprises changing a received frequency depending on variation of the frequency of the transmitted ultrasonic wave pulse.

To achieve the above object, according to the present invention, there is also provided a distance measurement method using ultrasonic waves, including the steps of: installing a first receiver for receiving an ultrasonic wave pulse at a known position; installing a second receiver for receiving an ultrasonic wave pulse at an object to be measured; transmitting an ultrasonic wave pulse having a specific frequency from a location where a distance from the object to be measured, to the first and second receivers; extracting specific frequencies of the ultrasonic wave pulses received from the first and second receivers to find an arrival time of a first pulse and converting the time into a distance; transmitting error information related to a difference between the distance received by the first receiver and the known distance to the second receiver; and allowing the second receiver to correct the velocity of sound using the error information.

To achieve the above object, according to the present invention, there is provided a distance measurement device using ultrasonic waves, including: a transmitter for generating an ultrasonic wave pulse having a specific frequency; a sensor for detecting the ultrasonic wave pulse reflected from an object; an amplifier for amplifying the ultrasonic wave pulse detected by the sensor; an analog filter for selectively attenuating other frequencies except for an specific frequency from the ultrasonic wave pulse amplified by the amplifier; a secondary amplifier for amplifying an analog signal selected through the analog filter; an A/D converter for converting the amplified analog signal to a digital data; a memory for storing the digital data therein; a digital signal processor for processing the digital data stored in the memory; an output unit for displaying results processed in the digital signal processor; a numerical input unit for informing the digital signal processor of a processing condition; and a communication unit for connecting the digital signal processor and an external apparatus to each other so that the digital signal processor and the external apparatus can exchange information, wherein a transmission time of a first pulse among the received ultrasonic wave pulse and a delayed time of an arrival time of the first pulse calculated in the digital signal processor are measured.

According to the present invention, a received ultrasonic wave signal is sufficiently amplified and a specific frequency is separated from an ultrasonic wave signal mixed with an unnecessary signal to extract an arrival signal of a first pulse. It is thus possible to calculate a distance.

An inventor of the present invention recognized that an ultrasonic wave pulse has a specific frequency and thus proposed the present invention. That is, a conventional distance measurement method using ultrasonic waves includes simply amplifying a received signal (pulse) and converting the signal into a distance. This method has a problem that an accurate distance cannot be measured depending on noise and the atmospheric state. In other words, the conventional threshold value detecting method using a signal of a predetermined amount has a limit to amplification of a signal due to noise since a signal can be measured when the amplitude of the signal is strong in a level of over the noise. Furthermore, a signal of the noise level is not measured. Thus, a signal which is initially weak cannot be detected because of the noise and cannot be thus discriminated from the noise. As a result, error in measurement increases as much as the number of the initial signal lost. In addition, a conventional envelope detecting method using the maximum amplitude requires a complicated process in which a differential gain must be different depending on the amount of a received signal since the amplitude of the received signal keeps constant. In this method, it is impossible to amplify the signal over a noise level.

Contrarily, in a state where a waveform is mixed with noise having a frequency different from a specific frequency of an ultrasonic wave pulse transmitted, the specific frequency of the ultrasonic wave pulse is separated and an arrival time of an ultrasonic wave pulse that is received among the separated ultrasonic wave pulses for the first time is converted to a distance. Therefore, in the distance measurement method using the ultrasonic waves according to the present invention, the fact that the frequency properties of noise and the ultrasonic wave signal are different is employed. Thus, it is possible to amplify a signal sufficiently together with noise without influence from the amount of noise and limit to amplification of the signal. Furthermore, noise and a weak ultrasonic wave signal are amplified at the same time. A specific frequency of an ultrasonic wave signal is separated from signals that are sufficiently strong and a first pulse is then restored from the specific frequency. Therefore, it is possible to measure a distance from an object by detecting an ultrasonic wave signal from a weak signal depending on long-distance measurement regardless of noise. In addition, since a maximum differential gain can be maintained regardless of the amount of a received signal, an device is simplified.

The present invention will now be described in detail in connection with preferred embodiments with reference to the accompanying drawings.

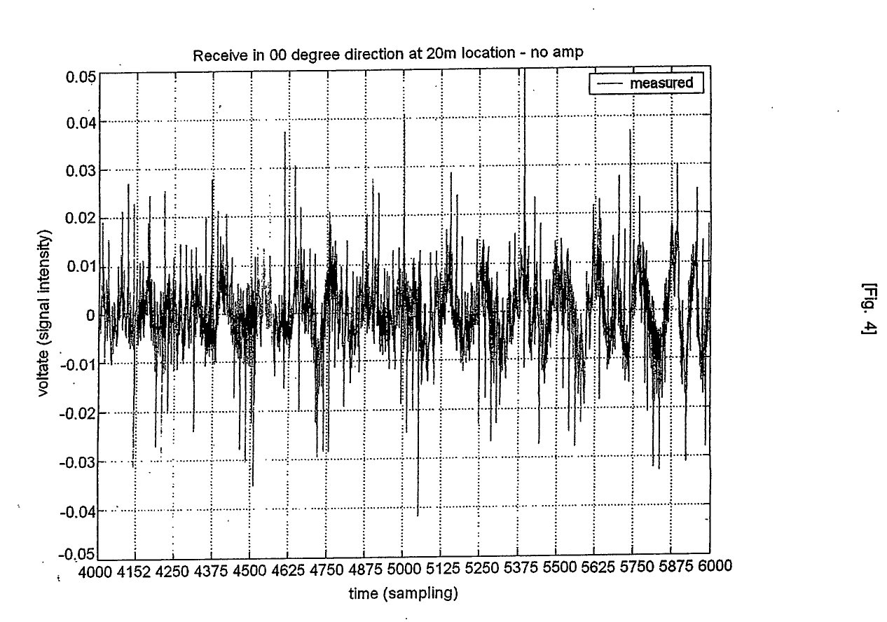

FIG. 3 sequentially shows a result of receiving ultrasonic waves, a convolution processing result and a result of analyzing a signal period for a received signal from the top by means of a distance measurement method using the period of an ultrasonic wave signal according to the present invention.

Referring to FIG. 3, in the present invention, the frequency may be separated by several methods such as a bandpass filter and moving averages. Ultrasonic waves contain the same period even in a received waveform, i.e., frequency components since a transmitted signal has a specific frequency. If the period is detected from the received signal, it is possible to detect the period more exactly regardless of the level of the amplitude. A signal that is actually received by a sensor contains noise signals of several frequency bands. In this situation, a method for detecting only a specific frequency may remove noise components effectively by using a bandpass filter whose bandwidth is very narrow. In its implementation, there is a mode in which an analog filter and a digital mode are used. The analog filter having a narrow bandwidth is accompanied by difficulties in manufacturing and adjustment. Therefore, in a preferred embodiment of the present invention, these problems have been overcome by using correlation between the sine wave of a specific frequency and a received waveform through a digital signal-processing mole. Through this, it is possible to obtain a result similar to the bandpass filter using convolution. It is also possible to determine a specific frequency easily. By doing so, there are effects in that specific frequency components only are amplified so that a signal of a specific frequency can be easily detected and signals of other frequency are relatively reduced.

### INDUSTRIAL APPLICABILITY

According to the present invention described above, the fact that the frequency properties of noise and an ultrasonic are different is employed. Thus, since it is not affected by the amount of noise, there is no limitation in amplification of a signal. It is thus possible to amplify the signal containing noise sufficiently.

Furthermore, noise and a weak ultrasonic signal are amplified together and a specific frequency of the ultrasonic signal is separated from a sufficiently strong signal and a first signal among the specific frequency is restored. Therefore, even in a weak signal depending on long-distance measurement, it is possible to measure an ultrasonic signal regardless of noise.

Moreover, it is possible to maintain a maximum differential gain regardless of the amount of a received signal. It makes the device simple. Therefore, the present invention has advantages in that long-distance measurement is possible since a weak signal can be amplified sufficiently and the accuracy can be increased since an initial signal lost can be restored.

1. A distance measurement method using ultrasonic, comprising the steps of:
transmitting an ultrasonic pulse having specific frequencies to each object;
receiving the ultrasonic pulse that is reflected from the object or directly transmitted; and
extracting a specific frequency of the received ultrasonic wave pulse to find an arrival time of a first pulse and converting the time into a distance.

2. The distance measurement method as claimed in claim 1, wherein the step of finding the arrival time and converting the time into the distance further includes the step of separating a specific frequency of the ultrasonic and converting an arrival time of an ultrasonic that is received for the first time among the separated ultrasonic into the distance, in a state where the waveform is mixed with noise having different frequency properties from the specific frequency of the transmitted ultrasonic.

3. The distance measurement method as claimed in claim 1, wherein in the step of converting the time into the distance, the extraction of the specific frequency from the received ultrasonic further comprises the steps of:
amplifying the received ultrasonic to generate an amplified signal;
weakening a signal of an unnecessary frequency among the amplified signal through an analog filter circuit to generate a filtered signal;
amplifying the filtered signal again to generated a re-amplified signal; converting the re-amplified signal into a digital signal; and
extracting a specific frequency from the converted digital signal through a digital signal processing.

4. The distance measurement method as claimed in claim 3, further comprising the step of specifying a distance range to be excluded when measuring a distance of the object, such that a distance exceeding the specified distance range is measured.

5. The distance measurement method as claimed in claim 1, wherein the step of receiving the ultrasonic reflected from the object while the object is moving comprises changing a received frequency depending on variation of the frequency of the transmitted ultrasonic.

6. A distance measurement method using ultrasonic, comprising the steps of:
installing a first receiver for receiving an ultrasonic at a known position;
installing a second receiver for receiving an ultrasonic at an object to be measured;
transmitting an ultrasonic having a specific frequency from a location where a distance from the object will be measured, to the first and second receivers;
extracting specific frequencies of the ultrasonic received from the first and second receivers to find an arrival time of a first signal and converting the time into a distance;
transmitting error information related to a difference between the distance received by the first receiver and the known distance to the second receiver; and
allowing the second receiver to correct the velocity of sound using the error information.

7. A distance measurement device using ultrasonic, comprising:
a transmitter for generating an ultrasonic having a specific frequency;
a sensor for detecting the ultrasonic reflected from an object;
an amplifier for amplifying the ultrasonic detected by the sensor;
an analog filter for selectively attenuating other frequencies except for a specific frequency from the ultrasonic amplified by the amplifier;
a secondary amplifier for amplifying an analog signal selected through the analog filter;
an A/D converter for converting the amplified analog signal to a digital data; a memory for storing the digital data therein;
a digital signal processor for processing the digital data stored in the memory; an output unit for displaying results processed in the digital signal processor;
a numerical input unit for informing the digital signal processor of a processing condition; and
a communication unit for connecting the digital signal processor and an external apparatus to each other so that the digital signal processor and the external apparatus can exchange information,
wherein a transmission time of a first signal among the received ultrasonic and a delayed time of an arrival time of the first signal calculated in the digital signal processor are measured.
